(19) United States
(12) Patent Application Publication
(10) Pub. No.: US 2014/0158789 A1
(43) Pub. Date: Jun. 12, 2014

(54) MEMBRANE AIR FRESHENER WITH DIRECT DOME LABLE

(71) Applicant: AMERICAN COVERS, INC., Draper, UT (US)

(72) Inventor: Bryce Haymond, Pleasant Grove, UT (US)

(73) Assignee: American Covers, Inc., Draper, UT (US)

(21) Appl. No.: 13/797,475

(22) Filed: Mar. 12, 2013

Related U.S. Application Data
(60) Provisional application No. 61/735,864, filed on Dec. 11, 2012.

(51) Int. Cl. A61L 9/12

(52) U.S. Cl. CPC A61L 9/12
USPC 239/34

(57) ABSTRACT

An air freshener includes a capsule having a permeable membrane extending across and covering a cavity, and a front surface. A fragrant material is disposed in the cavity of the vessel and is visible through a capsule wall. The fragrant material includes a scent permeating the permeable membrane. A recess is formed in the front surface of the vessel. An insert is coupled in the recess of the front surface with the insert carried directly by the capsule. Indicia is carried by the insert or the recess of the front surface. The air freshener has means for mounting the capsule.

### PRIORITY CLAIM(S)

Priority is claimed to U.S. Provisional Patent Application Ser. No. 61/735,864, filed Dec. 11, 2012, and which is hereby incorporated herein by reference.

### RELATED APPLICATION(S)/PATENT(S)

This is related to U.S. patent application Ser. No. 12/979,763, filed Dec. 28, 2010, which is hereby incorporated herein by reference.

This is related to U.S. patent application Ser. No. 12/979,601, filed Dec. 28, 2010, which is hereby incorporated herein by reference.

### BACKGROUND

1. Field of the Invention

The present invention relates generally to air fresheners.

2. Related Art

Various different types of air fresheners have been proposed. Some air fresheners include various different components including decorative housings and the like. Some air fresheners include complicated components and/or numerous components. Some air fresheners include materials that can damage surfaces.

### SUMMARY OF THE INVENTION

It has been recognized that it would be advantageous to develop an air freshener that is simple and can be used to promote products and/or businesses. In addition, it has been recognized that it would be advantageous to develop an air freshener with a volatile scent material that is configured to protect supporting surfaces from contact with the volatile material.

The invention provides an air freshener with a capsule having a vessel defined by a plastic sheet with an indentation therein forming a cavity circumscribed by a rim. The capsule has a permeable membrane extending across and covering the cavity, and affixed to the rim. The vessel has a front surface on a protrusion opposite to and defined by the indentation in the plastic sheet. The plastic sheet defines a vessel wall and is at least translucent with a substantially constant thickness. The protrusion matches the indentation on opposite sides of the vessel. A fragrant material is disposed in the cavity of the vessel and is visible through the vessel wall. The fragrant material includes a scent permeating the permeable membrane. A recess is formed in the front surface of the vessel. An insert is coupled in the recess of the front surface with the insert carried directly by the vessel. Indicia is carried by the insert or the recess of the front surface. The air freshener also has means for mounting the capsule attached to the vessel.

In addition, the invention provides an air freshener with a capsule having a vessel defined by a plastic sheet with an indentation therein forming a cavity circumscribed by a rim. The capsule has a permeable membrane extending across and covering the cavity and affixed to the rim. The vessel has a front surface on a protrusion opposite to and defined by the indentation in the plastic sheet. The plastic sheet is defines a vessel wall and is at least translucent with a substantially constant thickness. The protrusion matches the indentation on opposite sides of the vessel. A fragrant material is disposed in the cavity of the vessel and is visible through the vessel wall. The fragrant material includes a scent permeating the permeable membrane. A recess is formed in the front surface of the vessel. An insert is coupled in the recess of the front surface with the insert carried directly by the vessel. The insert includes: an adhesive film adhered to the front surface of the vessel; a polyurethane dome affixed to the adhesive film and adhered to the vessel by the adhesive film; and the dome having a broad, flat shape with an outfacing surface and with a flat and smooth center parallel with the adhesive film and surrounded by a radius perimeter. Indicia is carried by the insert or the recess of the front surface. The air freshener also has means for mounting the capsule attached to the vessel.

Furthermore, the invention provides an air freshener in combination with a vehicle air vent with a louver. The air freshener includes a capsule having a vessel with an indentation forming a cavity circumscribed by a rim. The capsule has a permeable membrane extending across the cavity and affixed to the rim to enclose the cavity, and a front surface on a protrusion opposite the permeable membrane. The vessel has a vessel wall that is at least translucent with a substantially constant wall thickness. The protrusion matches the indentation on opposite sides of the vessel. A fragrant material is disposed in the cavity of the vessel and visible through the vessel wall. The fragrant material includes a scent permeating the permeable membrane. A mount is rigidly affixed to a back of the rim opposite the front surface. The mount has a dimple therein. A standoff brace is rigidly affixed in the dimple of the mount. A clip is affixed to the standoff brace opposite the mount. The clip has a pair of fingers engaging the louver of the air vent and extending into the air vent with the permeable membrane separated from and spaced apart from the louver of the air vent by the standoff brace. At least a bottom portion of the mount abuts to the standoff brace to resist pivoting of the mount with respect to the standoff brace, and thus resist pivoting of the permeable membrane towards the louver. A recess is formed in the front surface of the vessel. An insert is coupled in the recess of the front surface. The insert includes: an adhesive film adhered to the front surface of the vessel; a polyurethane dome affixed to the adhesive film and adhered to the vessel by the adhesive film; and the dome having a broad, flat shape with an outfacing surface and with a flat and smooth center parallel with the adhesive film and surrounded by a radius perimeter. Indicia is carried by the insert or the recess of the front surface.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a perspective view of an air freshener in accordance with an embodiment of the present invention;

FIG. 2 is a front view of the air freshener of FIG. 1;

FIG. 3 is a side view of the air freshener of FIG. 1;

FIG. 4a is a cross-sectional side view of the air freshener of FIG. 1 taken alone line 4a of FIG. 2;

FIG. 4b is a cross-sectional side view of another air freshener in accordance with another embodiment of the present invention;

FIG. 5 is a perspective view of another air freshener in accordance with another embodiment of the present invention;

FIG. 6 is a top view of the air freshener of FIG. 5;

FIG. 7 is a side view of the air freshener of FIG. 5;

FIG. 8 is a front view of the air freshener of FIG. 5;

FIG. 9 is a rear view of the air freshener of FIG. 5;

FIG. 10 is a front view of another air freshener in accordance with another embodiment of the present invention;

FIG. 11 is an exploded view of an insert of the air freshener of FIG. 1; and

FIG. 12 is a partial cross-sectional side view of the insert of FIG. 11.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

### Definitions

The terms “scent” and “fragrance” are used interchangeably herein and include particular scents or even neutralizing agents. The terms “scent material” and “fragrant material” are used interchangeably herein and include the material that carries the scent or fragrance.

### Description

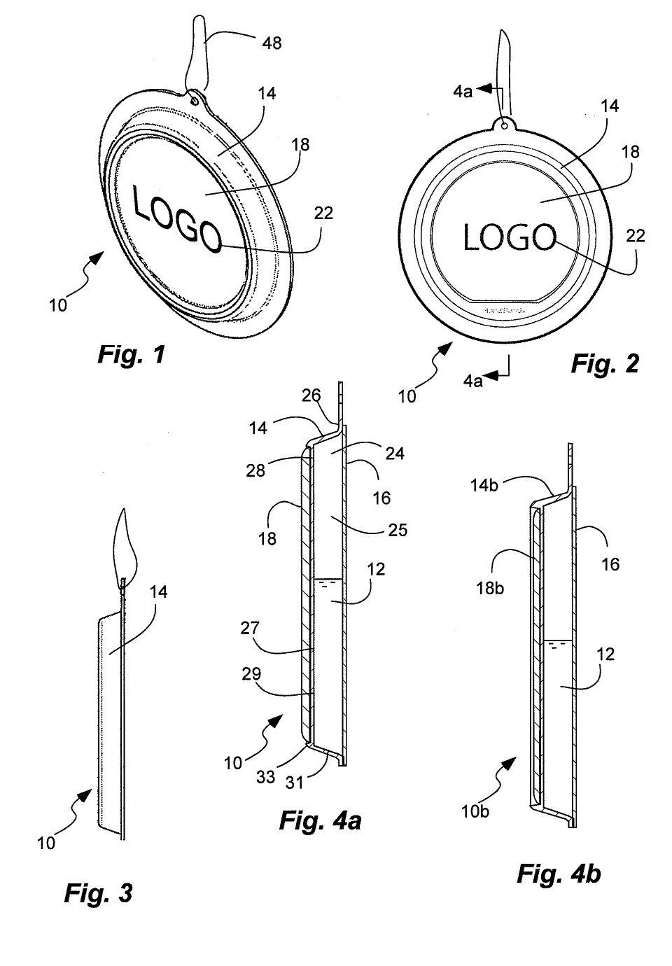

Referring to FIGS. 1-4a, an air freshener device, indicated generally at 10, in an example implementation in accordance with the invention is shown. The air freshener can be used in various different environments, such as bathrooms, wash closets, vehicles, offices, bedrooms, etc. In one aspect, the air freshener can be used in combination with a vent of an air duct through which air is forced or displaced. For example, the air freshener can be coupled to or suspended from a vehicle vent or louver, and used in combination with forced air through the vent to disperse a scent or fragrance. As another example, the air freshener can be hung in the vehicle, such as from the rear view mirror. The air freshener can provide a scent that can include, for example, floral, fruit, vanilla, berry, pine, etc. The scent can be provided by fragrant material 12, such as scented or fragrant oil.

The air freshener can have a simple design, such as capsule 14 with a permeable membrane 16 containing the fragrant material 12. The scent or fragrance can disperse or leach from the fragrant material or oil over time, and through the membrane 16; and the fragrant material or oil can reduce in mass or volume over time. The fragrant material 12 can be visible in or through the capsule 14 to determine the remaining amount of mass or volume. In addition, the air freshener 10 or capsule 14 can include an insert 18 with a logo or indicia 22 that is carried directly by the capsule 14, thus preserving the simplicity of the design. Thus, the air freshener 10 can be used as a promotional item of relatively low cost but also with relatively high value or perceived worth, and capable of placing the indicia 22 in front of a viewer for prolonged periods. The air freshener 10 can be provided by businesses, corporations or other organizations, with the indicia 22 indicative of or representing a business (or company or organization), a product or service, or both, such as a logo, message, slogan, etc. The indicia 22 can be or can include letters, symbols, figures, pictures, logos, art, corporate messages, slogans, bi-lines, icons, etc. that are associated with or that form a representation of a business, company or organization or the like, or a product, service or the like, or both.

The scent material 12 can be provided in a cartridge or capsule 14. The capsule 14 can have, and the scent material 12 can be contained in, a vessel 24 of the capsule, or formed by the capsule and the membrane. The capsule 14, and vessel 24 therein, can be formed by or defined by a plastic sheet with an indentation 25 therein forming a cavity circumscribed by a rim 26. The indentation in the plastic sheet forms the cavity on one side of the sheet, and a protrusion 27 on the opposite side of the sheet that is defined by and matches the indentation on the opposite side. Thus, the indentation and protrusions are on opposite sides of the sheet, and the capsule. The protrusion can define a front of the capsule or vessel, while the indentation can define a rear of the capsule or vessel. The capsule or vessel or protrusion has a front surface 29. The plastic sheet defines a capsule or vessel wall 31 with a substantially constant thickness, and the plastic sheet or wall can be transparent or at least translucent, or have a portion thereof that is transparent or translucent. Thus, the fragrant material 12 can be visible through the wall 31, and thus through the capsule 14 and vessel 24. The capsule or vessel or cavity thereof can be formed by vacuum forming the plastic sheet, and die cutting the capsule or vessel from the vacuum formed plastic sheet.

The permeable membrane 16 is disposed over and covers and extends across the cavity or indentation 25, defining the vessel between the plastic sheet of the capsule and the permeable membrane. Thus, the vessel 24 is formed by and completely enclosed by the plastic sheet and the permeable membrane. The fragrance or scent of the fragrant material 12 can permeate through the permeable membrane 16 over time. The permeable membrane 16 can be planar or flat, and can define a facing direction of the capsule or vessel as the surface or operable surface through which the scent permeates. The air freshener 10 or capsule 14 can be provided with a release liner that is disposed over the membrane prior to use and releasable therefrom to initiate use. The release liner can have a tacky or adhesive surface or perimeter that adheres to the membrane or rim. The release liner can be a foil that covers the membrane, and is temporarily sealed around its perimeter. This foil prevents the fragrance from evaporation prior to usage. When the customer receives the product, they remove the foil covering the membrane, and that begins the scent release. The vessel or capsule, or plastic sheet or wall thereof, can be transparent or translucent, and the fragrant material 12 can have a color so that the fragrant material is visible through the transparent or translucent vessel or capsule, or wall thereof. The permeable membrane 16 can also be transparent or translucent so that light can shine through the membrane, and the fragrant material, and through the vessel to illuminate the fragrant material. In one aspect the fragrant material is scented oil. In another aspect, the fragrant material is a scented gel. In one aspect, the fragrant material is at least translucent. In another aspect, the fragrant material can be opaque.

The front surface 29 of the capsule 14 or vessel 24 or protrusion 27 can include a recess 28 formed therein. The recess 28 can be the same shape as the capsule, vessel or protrusion, and can be broad and flat. An annular lip 33 can circumscribe the recess 28 and extend forwardly. The insert 18 can be disposed in and coupled in, or partially within, the recess 28. In one aspect, the insert 18 can extend out of the recess 28 for greater visibility, as shown in FIG. 4a. In another aspect, an air freshener 10b can have a capsule 14b or vessel with an insert 18b recessed within recess 28 or below the annular lip thereof to protect the insert, as shown in FIG. 4b. In another aspect, the insert can be flush with the recess or annular lip thereof for both visibility and protection.

The insert 18 can carry indicia 22, such as a logo. In one aspect, the insert or a portion or majority thereof, can be transparent or translucent, and the indicia can be formed on or printed on an inner surface of the insert so that the indicia is visible through the insert. Thus, the insert can carry the indicia and protect the indicia. In another aspect, the indicia can be disposed on or printed on a surface of the recess of the capsule so that the indicia is visible through the insert. Thus, the insert can protect the indicia. Thus, the indicia 22 can be carried by either the insert or the capsule or vessel, such as on the front surface in the recess. In another aspect, the insert can be opaque and the indicia can be formed on or printed on the insert.

Referring to FIGS. 11 and 12, the insert 18, and/or the recess, can include an adhesive film 30 adhered between the insert and the recess or the capsule or vessel thereof to retain the insert in the recess, and retain the insert to the capsule or vessel. The adhesive film can use a chemical adhesive to adhere. The adhesive can also be selected so that the pad can be removed if desired. Thus, the adhesive film can include a releasable adhesive, as opposed to a permanent adhesive. The adhesive film can be a repositionable adhesive film releasably and repositionably adhered to the insert and/or the recess of the capsule. The adhesive film can have an adhesive bond between the insert and the recess of the capsule. The adhesive and/or the adhesive film can be substantially transparent, or at least translucent, so that the indicia and/or fragrant material can be visible through the adhesive film. The adhesive and/or adhesive layer can be a removable 3M Controltac™ adhesive.

The adhesive film 30 can be disposed on a substrate 34. The substrate 34 can also be a film. In one aspect, the substrate can be substantially non-absorbent. The substrate can provide a surface, such as an upper or inner surface opposite the adhesive that can be printable. In addition, the substrate can be substantially transparent, or at least translucent, so that the indicia and/or fragrant material can be visible through the insert. The substrate can be a printed 3M Graphics Vinyl. Alternatively, the substrate can be a polyester film. The substrate can be any laminated (non-porous) substrate, including: PP, PVC, PET, ABS, polyester, regular sticker paper, vinyl, or tin metal (foil). Alternatively, the substrate can have be other films with other characteristics, such as reflective, metal shine, textured, etc.

A dome 38 can be affixed to the adhesive film 30 and/or substrate 34, and adhered to the recess by the adhesive film. The dome 38 is secured to the adhesive film and/or substrate. The dome can have a broad, flat, squat shape with an outfacing surface 42 that faces outwardly. The outfacing surface can be smooth and flat and parallel with the adhesive layer, and the recess. In addition, the outfacing surface can be surrounded by a radius perimeter 44. Thus, the perimeter of the outfacing surface curves toward the adhesive layer and recess. In one aspect, all of the outfacing surface 42 can be flat and smooth and continuous between the radiused perimeter. The dome 38 can be substantially transparent, or at least translucent, so that the indicia and/or fragrant material can be visible through the dome and the insert. The dome can be formed of polyurethane. The dome between the radius perimeter can be flat and smooth and continuous to accommodate the indicia. The broad face 42 provides a greater surface area to allow for more and/or larger indicia.

As described above, the dome can be formed of polyurethane. The polyurethane can be formed by combining a polyol with a catalyst or hardener. In one aspect, the ratio of polyol to catalyst can be between 0.63 to 0.85, so that there is more polyol than catalyst. In another aspect, the ratio of polyol to catalyst can be between 0.63 to 0.75. Typical polyurethane is obtained by mixing polyol and catalyst in a 1:1 ratio. In addition, in one aspect, the outfacing surface 42 or dome 38 can have a hardness between 10 and 35 Shore D. In another aspect, the outfacing surface 42 or dome 38 can have a hardness between 12 and 21 Shore D. In another aspect, the outfacing surface 34 or dome 30 can have a hardness between 12 and 18 Shore D. In another aspect, the outfacing surface 42 or dome 38 can have a hardness of 60 Shore D or harder.

The indicia 22 can include a graphic layer that can be disposed between the adhesive film 30 and the dome 38, or between the substrate 34 and the dome 38. The graphic layer can include ink disposed on or printed on the substrate. For example, the graphic layer can be an aqueous based inkjet ink. The graphic layer can include indicia, such as a logo, a picture, etc. In addition, the graphic layer can include directions for use, orientation, care, etc. Furthermore, the graphic layer can be or can include other inclusions. The graphic layer can be visible through the dome 38. The graphic layer and/or inclusion can be smaller or can have a smaller size than the substrate and dome such that the graphic layer and inclusion are surrounded by a perimeter of the dome and substrate.

As indicated above, the graphic layer can be or can include an inclusion. The inclusions can be embedded in the dome. For example, the graphic layer can be an etched metallic layer. The metallic layer can be etched to form the indicia. Other inclusions can be an LED (or other light source) and a battery power source; a hologram or lenticular artwork; an RFID computer chip or tag; a mirror; one or more gems or faux gems; etc. For example, an LED can be electrically coupled to a battery. The LED may remain continuously activated or lit. Alternatively, the LED may be activated, or the leads for the LED selectively contacted by the battery, such as by pressure applied to the dome or insert. Alternatively, a light sensor can be electrically coupled between the battery and the LED to activate the LED in low light conditions. Other electronics can be included for special effects, such as flashing, etc. It will be appreciated that multiple lights or LEDs of one or more color can be included or embedded in the dome. As another example, a gem or faux gem can be embedded in the dome. The inclusion(s) can be disposed on the adhesive film and/or substrate, and surrounded by the material of the dome so that the inclusion projects into the dome. Thus, the inclusions can be inclusions for the dome and/or the graphics layer.

The dome can also include inclusions embedded therein. For example, the dome can include a scented material, such as a scented oil, that is permeable through the polyurethane material of the dome. In addition, the dome can be colored and translucent. Furthermore, the dome can include a florescent material.

In another aspect, the insert or dome can be opaque with the indicia and/or graphic layer, such as printing, on the outfacing surface. The opaque dome or insert can have a color to match or complement the fragrant material.

The insert can be made by pouring the polyurethane material on top of the substrate and graphic layer to form the dome. Thus, the radius perimeter is obtained by individual pouring each insert or dome. The graphic layer can be printed on the substrate having the adhesive film opposite the graphic layer. The graphic layer can be a discrete graphic contained within the insert or dome, as opposed to a continuous graphic.

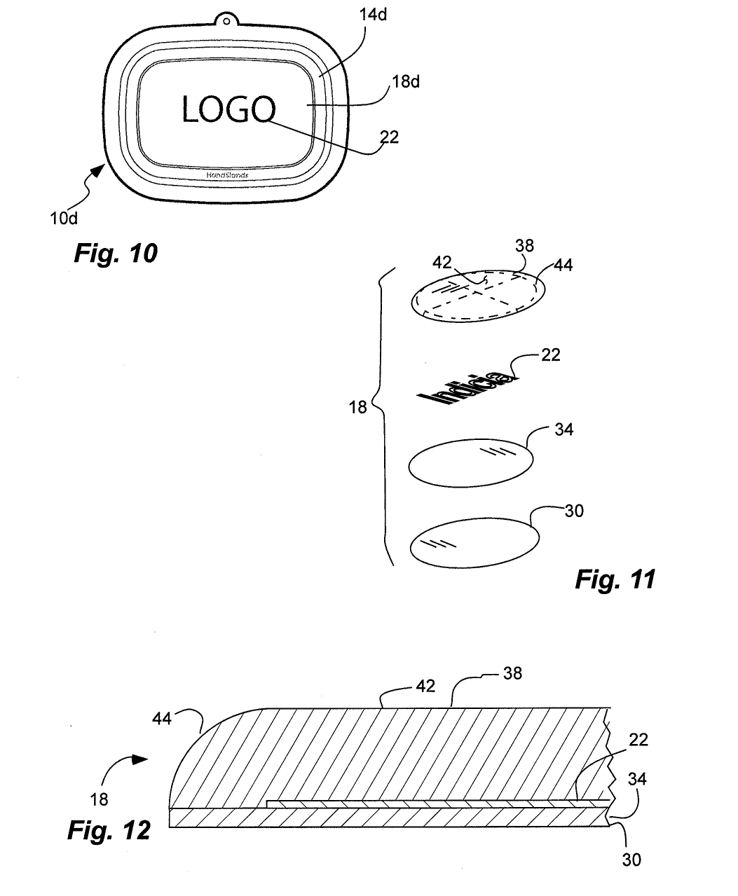

In one aspect, the insert 18 can have a round shape, as shown in FIGS. 1-9. The round shape can provide an efficient package. In another aspect, an air freshener 10d, the capsule 14d and the insert 18d thereof can have a rectangular shape with rounded corners, as shown in FIG. 10. The rectangular shape can accommodate more or larger indicia. Alternatively, other shapes are possible.

Referring again to FIGS. 1-4a, the air freshener 10 can include a hanger, such as a loop of string 48, attached to the rim of the capsule or vessel to hang, suspend or mount the air freshener. The term “mount” is used herein to including hanging, suspending or the like. The hanger or loop is an example of one means for mounting the capsule attached to the vessel. Other such means include by way of example clips, etc. The hanger forms a single point connection to the rim. Thus, the capsule, the vessel and the rim are exposed and free from other structure, except for the single point connection and the insert. Thus, the structure of the air freshener is simplified, and the common decorative housing or body is eliminated. The hanger or loop of string can hang or suspend the air freshener in a vehicle, such as from the rear view mirror.

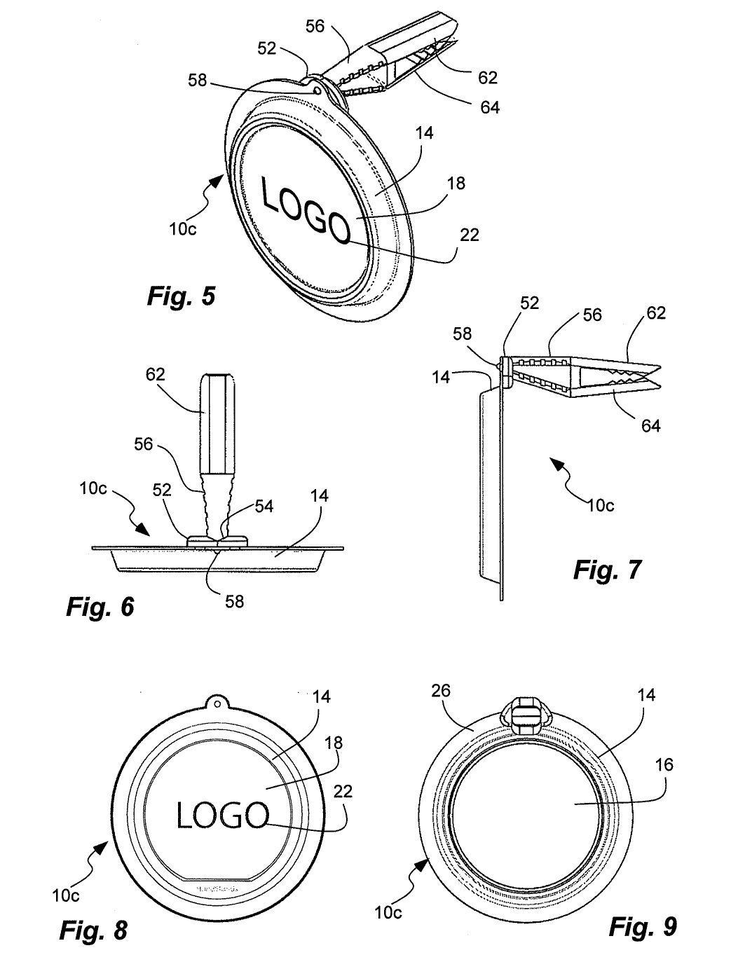

Referring to FIGS. 5-9, another air freshener 10b is shown that is similar in many respects to that described above, and which description is herein incorporated by reference. The air freshener 10b includes a mount 52 rigidly affixed to a back of the rim 26 opposite the front surface. The mount 52 can have a dimple 54 therein on the back thereof. A front of the mount 52 can be flat and can abut to the rim. The mount forms a single point connection. A standoff brace 56 is rigidly affixed in the dimple 54 of the mount 52, and extends therefrom. The standoff brace can have a finger 58 with an enlarged distal end or head can press fit through an aperture in the mount and an aperture in the rim to secure the standoff brace, and the mount, to the rim. A clip 62 is affixed to the standoff brace 56 opposite the mount 52. The clip 62 includes a pair of fingers 64 configured to engage a louver of an air vent and extend into the air vent. The standoff brace 56 maintains the permeable membrane 16 separated from and spaced apart from the louver of the air vent. Thus, any of the fragrant material leaching through the membrane does not contact the louver. At least a bottom portion of the mount abuts to the standoff brace to resist pivoting of the mount with respect to the standoff brace, and thus resist pivoting of the permeable membrane towards the louver. The mount is another example of means for mounting the capsule attached to the vessel.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. An air freshener device, comprising:
a) a capsule having a vessel defined by a plastic sheet with an indentation therein forming a cavity circumscribed by a rim, and a permeable membrane extending across and covering the cavity and affixed to the rim, the vessel having a front surface on a protrusion opposite to and defined by the indentation in the plastic sheet;
b) the plastic sheet defining a vessel wall and being at least translucent with a substantially constant thickness, and with the protrusion matching the indentation on opposite sides of the vessel;
c) a fragrant material disposed in the cavity of the vessel and visible through the vessel wall, the fragrant material including a scent permeating the permeable membrane;
d) a recess formed in the front surface of the vessel;
e) an insert coupled in the recess of the front surface with the insert carried directly by the vessel;
f) indicia carried by the insert or the recess of the front surface; and
g) means for mounting the capsule attached to the vessel.

2. A device in accordance with claim 1, wherein the means for mounting forms a single point connection to the rim; and wherein the capsule, the vessel and the rim are exposed and free from other structure, except for the single point connection and the insert.

3. A device in accordance with claim 1, wherein the insert extends beyond the recess in the front surface of the vessel.

4. A device in accordance with claim 1, wherein the insert is recessed within the recess in the front surface of the vessel.

5. A device in accordance with claim 1, wherein the insert further comprises:
a dome coupled to the front surface and having a broad, flat shape with an outfacing surface and with a flat and smooth center parallel to the front surface and surrounded by a radius perimeter.

6. A device in accordance with claim 5, wherein the dome is at least translucent; and
wherein the indicia is disposed between the dome and the vessel, and visible through the dome.

7. A device in accordance with claim 5, wherein the dome is opaque; and wherein the indicia is disposed on the outfacing surface of the dome.

8. A device in accordance with claim 5, wherein the insert further comprises:
a) an adhesive film adhered to the front surface of the vessel;
b) a polyurethane dome affixed to the adhesive film and adhered to the vessel by the adhesive film.

9. A device in accordance with claim 8, wherein the dome is translucent or transparent; and further comprising a graphic layer disposed between the adhesive film and the dome and visible through the dome.

10. A device in accordance with claim 9, further comprising:
a film with the adhesive film on one side; and
the graphic layer printed on the other side.

11. A device in accordance with claim 9, further comprising:
a substrate with the adhesive film on one side and the graphic layer printed on the other side; and
the substrate is at least translucent so that the entire insert except for the graphic layer is translucent or transparent.

12. A device in accordance with claim 5, wherein all of the outfacing surface of the dome between the radius perimeter is flat and smooth and continuous.

13. A device in accordance with claim 1, wherein the means for mounting further comprises:
a) a mount rigidly affixed to a back of the rim opposite the front surface and having a dimple therein;
b) a standoff brace rigidly affixed in the dimple of the mount; and
c) a clip affixed to the standoff brace opposite the mount.

14. A device in accordance with claim 13, wherein the clip further comprises:
a pair of fingers configured to engage a louver of an air vent and extend into the air vent with the permeable membrane separated from and spaced apart from the louver of the air vent by the standoff brace and with at least a bottom portion of the mount abutting to the standoff brace to resist pivoting of the mount with respect to the standoff brace, and thus resist pivoting of the permeable membrane towards the louver.

15. An air freshener device, comprising:
a) a capsule having a vessel defined by a plastic sheet with an indentation therein forming a cavity circumscribed by a rim, and a permeable membrane extending across and covering the cavity and affixed to the rim, the vessel having a front surface on a protrusion opposite to and defined by the indentation in the plastic sheet;
b) the plastic sheet defining a vessel wall and being at least translucent with a substantially constant thickness, and with the protrusion matching the indentation on opposite sides of the vessel;
c) a fragrant material disposed in the cavity of the vessel and visible through the vessel wall, the fragrant material including a scent permeating the permeable membrane;
d) a recess formed in the front surface of the vessel;
e) an insert coupled in the recess of the front surface with the insert carried directly by the vessel, the insert including:
i) an adhesive film adhered to the front surface of the vessel;
ii) a polyurethane dome affixed to the adhesive film and adhered to the vessel by the adhesive film; and
iii) the dome having a broad, flat shape with an outfacing surface and with a flat and smooth center parallel with the adhesive film and surrounded by a radius perimeter;
f) indicia carried by the insert or the recess of the front surface; and
g) means for mounting the capsule attached to the vessel.

16. A device in accordance with claim 15, wherein the means for mounting forms a single point connection to the rim; and wherein the capsule, the vessel and the rim are exposed and free from other structure, except for the single point connection and the insert.

17. A device in accordance with claim 15, wherein the dome is translucent or transparent; and further comprising a graphic layer disposed between the adhesive film and the dome and visible through the dome.

18. A device in accordance with claim 17, further comprising:
a film with the adhesive film on one side; and
the graphic layer printed on the other side.

19. A device in accordance with claim 17, further comprising:
a substrate with the adhesive film on one side and the graphic layer printed on the other side; and
the substrate is at least translucent so that the entire insert except for the graphic layer is translucent or transparent.

20. A device in accordance with claim 15, wherein the means for mounting further comprises:
a) a mount rigidly affixed to a back of the rim opposite the front surface and having a dimple therein;
b) a standoff brace rigidly affixed in the dimple of the mount; and
c) a clip affixed to the standoff brace opposite the mount.

21. An air freshener device in combination with a vehicle air vent with a louver, the device comprising:
a) a capsule having a vessel with an indentation forming a cavity circumscribed by a rim, and a permeable membrane extending across the cavity and affixed to the rim to enclose the cavity, and a front surface on a protrusion opposite the permeable membrane;
b) the vessel having a vessel wall that is at least translucent with a substantially constant wall thickness, and with the protrusion matching the indentation on opposite sides of the vessel;
c) a fragrant material disposed in the cavity of the vessel and visible through the vessel wall, the fragrant material including a scent permeating the permeable membrane;
d) a mount rigidly affixed to a back of the rim opposite the front surface and having a dimple therein;
e) a standoff brace rigidly affixed in the dimple of the mount;
f) a clip affixed to the standoff brace opposite the mount having a pair of fingers engaging the louver of the air vent and extending into the air vent with the permeable membrane separated from and spaced apart from the louver of the air vent by the standoff brace and with at least a bottom portion of the mount abutting to the standoff brace to resist pivoting of the mount with respect to the standoff brace, and thus resist pivoting of the permeable membrane towards the louver;
g) a recess formed in the front surface of the vessel;
h) an insert coupled in the recess of the front surface, the insert including:
i) an adhesive film adhered to the front surface of the vessel;
ii) a polyurethane dome affixed to the adhesive film and adhered to the vessel by the adhesive film; and
iii) the dome having a broad, flat shape with an outfacing surface and with a flat and smooth center parallel with the adhesive film and surrounded by a radius perimeter;
i) indicia carried by the insert or the recess of the front surface.
